(19) United States
(12) Patent Application Publication
(10) Pub. No.: US 2026/0274932 A1
(43) Pub. Date: Sep. 17, 2026

(54) ANTIBODY FORMULATION

(71) Applicant: Hoffmann-La Roche Inc., Little Falls, NJ (US)

(72) Inventors: Andrea ALLMENDINGER, Grenzach-Wyhlen (DE); Stefan FISCHER, Basel (CH)

(73) Assignee: Hoffmann-La Roche Inc., Little Falls, NJ (US)

(21) Appl. No.: 19/565,169

(22) Filed: Mar. 12, 2026

Related U.S. Application Data
(63) Continuation of application No. PCT/EP2024/075456, filed on Sep. 12, 2024.

(30) Foreign Application Priority Data
Sep. 14, 2023 (EP) 23197287.8

(51) Int. Cl. C07K 16/18; A61K 47/18; A61K 47/20; A61K 47/22; A61K 47/26; A61K 47/34

(52) U.S. Cl. CPC C07K 16/18; A61K 47/183; A61K 47/20; A61K 47/22; A61K 47/26; A61K 47/34

(57) ABSTRACT

This invention relates to a pharmaceutical formulation of an alpha synuclein antibody.

### CROSS-REFERENCE TO RELATED APPLICATIONS

This application is a continuation of International Application No. PCT/EP2024/075456 filed Sep. 12, 2024, which claims the benefit of and priority to European Patent Application No. 23197287.8, filed Sep. 14, 2023, each of which is incorporated herein by reference in its entirety.

### REFERENCE TO AN ELECTRONIC SEQUENCE LISTING

The instant application contains a Sequence Listing submitted electronically in XML format and is hereby incorporated herein by reference in its entirety. Said XML copy, created on Feb. 22, 2026, is named P35397-US-1_Sequence_Listing_xml, and is 5,315 bytes in size.

The present invention relates to a pharmaceutical formulation of an alpha synuclein antibody.

Parkinson's disease (PD) is a slow, chronic, progressive neurodegenerative estimated to affect between 7-10 million people worldwide. In the United States, an estimated 725,000 people are affected and more than 50,000 new cases are reported every year. Although 5 to 10 percent of patients are diagnosed before age 50, PD is generally considered a disease that targets older adults, affecting one out of every 100 people over the age of 60, and it is more common in men than in women.

Alpha-synuclein is a protein that is normally associated with synapses and is believed to play a role in neural plasticity, learning and memory. Alpha-synuclein can aggregate to form insoluble fibrils in pathological conditions, and is a major component of pathology that characterizes several neurodegenerative disorders including Parkinson's disease. Soluble oligomers of alpha-synuclein may be neurotoxic. The accumulation of alpha-synuclein with similar morphological and neurological alterations in species and animal models as diverse as humans, mice, and flies suggests that this molecule contributes to the development of Parkinson's disease. Antibodies directed against alpha-synuclein may be able to reduce alphasynuclein deposits and symptoms in Parkinson's disease.

Antibody molecules, as part of the group of protein pharmaceuticals, are very susceptible to physical and chemical degradation. Chemical degradation includes any process that involves modification of the protein via bond formation or cleavage, yielding a new chemical entity. A variety of chemical reactions is known to affect proteins. These reactions can involve hydrolysis including cleavage of peptide bonds as well as deamidation, isomerization, oxidation and decomposition. Physical degradation refers to changes in the higher order structure and includes denaturation, adsorption to surfaces, aggregation and precipitation. Protein stability is influenced by the characteristics of the protein itself, e.g. the amino acid sequence, the glycosylation pattern, and by external influences, such as temperature, solvent pH, excipients, interfaces, or shear rates. So, it is important to define the optimal formulation conditions to protect the protein against degradation reactions during manufacturing, storage and administration. (Manning, M. C., et al. (1989), “Stability of protein pharmaceuticals”, Pharm Res 6 (11), 903-918; Zheng, J. Y., Janis, L. J. (2005), “Influence of pH, buffer species, and storage temperature on physicochemical stability of a humanized monoclonal antibody LA298”, Int. J. Pharmaceutics 308, 46-51). Stable liquid formulations of therapeutic antibodies are particularly difficult to obtain when the formulation should include antibodies in a high concentration.

It is therefore an object of the present invention to provide a stable formulation for an alpha synuclein antibody with as few as necessary excipients, which enables the desired dosing and allows convenient administration of the antibody to a patient.

### SUMMARY

In a first aspect, the invention relates to a pharmaceutical formulation comprising:
120 to 200 mg/ml of an alpha synuclein antibody;
1 to 50 mM of a buffering agent;
0.01 to 1% (w/v) of a surfactant;
1 to 50 mM of at least one stabilizer;
100-150 mM tonicity agent,
at a pH in the range of from 5.0 to 7.0.

In an embodiment of the present invention, the anti-syunclein antibody concentration is in the range of 150 to 200 mg/ml, preferably 180 mg/ml.

In an embodiment of the present invention, the buffering agent is a histidine buffer, preferably a histidine acetate buffer.

In an embodiment of the present invention, the buffering agent is at a concentration of 10 to 40 mM.

In an embodiment of the present invention, the buffering agent provides a pH of 5.0 to 6.0, preferably 5.5.

In an embodiment of the present invention, the surfactant is a polysorbate, preferably a polysorbate 20.

In an embodiment of the present invention, the surfactant is at a concentration of 0.01 to 0.1% (w/v).

In an embodiment of the present invention, at least one stabilizer is selected from the group of salts, saccharides, and amino acids, preferably histidine.

In an embodiment of the present invention, the at least one stabilizer is at a concentration of 5 to 50 mM.

In an embodiment of the present invention, the tonicity agent is at a concentration of 120-150 mM, preferably 130 mM.

In an embodiment of the present invention, the tonicity agent is Arginine-HCl.

In an embodiment of the present invention, the formulation comprises:
150 to 180 mg/ml alpha synuclein antibody;
15 to 30 mM L-histidine/acetate buffer;
0.02 to 0.05% (w/v) polysorbate 20;
120 to 180 mM arginine-HCl;
5 to 25 mM L-methionine;
at a pH of 5.5±0.5.

In an embodiment of the present invention, the formulation comprises:
180 mg/ml alpha synuclein antibody,
20 mM L-histidine/acetate buffer,
0.04% (w/v) polysorbate 20,
130 mM arginine-HCl,
10 mM methionine, at pH 5.5;

In an embodiment of the present invention, the anti-alpha synuclein antibody comprises a VH domain having the amino acid sequence of Seq. Id. No. 1; and VL domain having the amino acid sequence of Seq. Id. No. 2.

In an embodiment of the present invention, the alpha synuclein antibody comprises a heavy chain having the amino acid sequence of Seq. Id. No. 3 and a light chain having amino acid sequence of Seq. Id. No. 4.

In an embodiment of the present invention, the alpha synuclein antibody is the antibody with the INN prasinezumab.

In an embodiment of the present invention, the formulation is in a liquid form, in a lyophilized form or in a liquid form reconstituted from a lyophilized form.

### DETAILED DESCRIPTION OF THE INVENTION

The term “buffering agent” as used herein denotes a pharmaceutically acceptable excipient, which stabilizes the pH of a pharmaceutical preparation. Suitable buffers are well known in the art and can be found in the literature. For example, citrate salts, acetate salts, histidine salts, succinate salts, malate salts, phosphate salts or lactate salts, and/or the respective free acids or bases thereof, as well as mixtures of the various salts and/or acids and bases thereof can be employed. Preferred pharmaceutically acceptable buffers comprise but are not limited to histidine buffers, citrate buffers, succinate buffers, acetate buffers and phosphate buffers. Preferred buffers for use in the present invention are histidine buffers, i.e. buffers having histidine, generally L-histidine, as buffering agent. Most preferred is L-histidine/HCl buffer, comprising L-histidine or mixtures of L-histidine and L-histidine hydrochloride and pH adjustment achieved with hydrochloric acid. Unless otherwise indicated, the term “L-histidine” when used herein to describe a buffering agent, refers to L-histidine/HCl buffer. L-histidine/HCl buffer can be prepared by dissolving suitable amounts of L-histidine and L-histidine hydrochloride in water, or by dissolving a suitable amount of L-histidine in water and adjusting the pH to the desired value by addition of hydrochloric acid. The abovementioned buffers are generally used at a concentration of about 1 mM to about 100 mM, preferably of about 10 mM to about 50 mM, more preferably of about 15 to 30 mM, and most preferably of 20 mM. Regardless of the buffer used, the pH can be adjusted to a value in the range from about 4.0 to about 7.0, preferably about 5.0 to about 6.0, and most preferably about 5.5, with an acid or a base known in the art, e.g. hydrochloric acid, acetic acid, phosphoric acid, sulfuric acid and citric acid, sodium hydroxide and potassium hydroxide.

A “stable” formulation is one in which the protein therein, e.g. the antibody, essentially retains its physical and chemical stability and thus its biological activity upon storage.

A “stable liquid pharmaceutical antibody formulation” is a liquid antibody formulation with no significant changes observed at a refrigerated temperature (2-8° C.) for at least 12 months, particularly 2 years, and more particularly 3 years. The criteria for stability are the following: no more than 10%, particularly 5%, of antibody monomer is degraded as measured by size exclusion chromatography (SEC-HPLC). Furthermore, the solution is colorless or clear to slightly opalescent by visual analysis. The protein concentration of the formulation has no more than +/−10% change. No more than 10%, particularly 5% of aggregation is formed. The stability is measured by methods known in the art such UV spectroscopy, size exclusion chromatography (SEC-HPLC), Ion-Exchange Chromatography (IE-HPLC), turbidimetry and visual inspection.

The term “surfactant” as used herein denotes a pharmaceutically acceptable, surface-active agent. Preferably, a non-ionic surfactant is used. Examples of pharmaceutically acceptable surfactants include, but are not limited to, polyoxyethylen-sorbitan fatty acid esters (Tween), polyoxyethylene alkyl ethers (Brij), alkylphenylpolyoxyethylene ethers (Triton X), polyoxyethylene-polyoxypropylene copolymers (Poloxamer, Pluronic), and sodium dodecyl sulphate (SDS). Preferred polyoxyethylene-sorbitan fatty acid esters are polysorbate 20 (polyoxyethylene sorbitan monolaureate, sold under the trademark Tween 20™) and polysorbate 80 (polyoxyethylene sorbitan monooleate, sold under the trademark Tween 80™). Preferred polyethylene-polypropylene copolymers are those sold under the names Pluronic® F68 or Poloxamer 188™. Preferred polyoxyethylene alkyl ethers are those sold under the trademark Brij™. Preferred alkylphenylpolyoxyethylene ethers are sold under the tradename Triton X, most preferred is p-tert-octylphenoxy polyethoxyethanol (sold under the tradename Triton X-100™). Preferred surfactants for use in the present invention are polyoxyethylen-sorbitan fatty acid esters, preferably polysorbate 20 or polysorbate 80, most preferably polysorbate 20. When polysorbate 20 (Tween 20™) and polysorbate 80 (Tween 80™) are used, they are generally used at a concentration range of about 0.001 to about 1%, preferably of about 0.01 to about 0.1%, more preferably of about 0.02% to about 0.05%, most preferably of about 0.05%. In the formulation of the invention, the concentration of the surfactant is described as a percentage, expressed in weight/volume (w/v).

The term “stabilizer” as used herein denotes a pharmaceutically acceptable excipient, which protects the active pharmaceutical ingredient and/or the formulation from chemical and/or physical degradation during manufacturing, storage and application. Stabilizers include but are not limited to saccharides, amino acids, polyols (e.g. mannitol, sorbitol, xylitol, dextran, glycerol, arabitol, propylene glycol, polyethylene glycol), cyclodextrines (e.g. hydroxypropyl-β-cyclodextrine, sulfobutylethyl-β-cyclodextrine, β-cyclodextrine), polyethylenglycols (e.g. PEG 3000, PEG 3350, PEG 4000, PEG 6000), albumines (e.g. human serum albumin (HSA), bovine serum albumin (BSA)), salts (e.g. sodium chloride, magnesium chloride, calcium chloride), chelators (e.g. EDTA) as hereafter defined. As mentioned hereinabove, stabilizers can be present in the formulation in an amount of about 1 to about 500 mM, preferably in an amount of about 10 to about 300 mM and more preferably in an amount of about 120 mM to about 300 mM. More than one stabilizer, selected from the same or from different groups, can be present in the formulation.

The term “saccharide” as used herein includes monosaccharides and oligosaccharides. A monosaccharide is a monomeric carbohydrate which is not hydrolysable by acids, including simple sugars and their derivatives, e.g. aminosugars. Saccharides are usually in their D conformation. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a linear chain. The monomeric saccharide units within an oligosaccharide can be identical or different. Depending on the number of monomeric saccharide units the oligosaccharide is a di-, tri-, tetra-penta- and so forth saccharide. In contrast to polysaccharides the monosaccharides and oligosaccharides are water soluble. Examples of oligosaccharides include sucrose, trehalose, lactose, maltose and raffinose. Preferred saccharides for use in the present invention are sucrose and trehalose (i.e. α,α-D-trehalose), most preferred is sucrose. Trehalose is available as trehalose dihydrate. Saccharides can be present in the formulation in an amount of about 100 to about 500 mM, preferably in an amount of about 200 to about 300 mM, more preferably in an amount of about 220 to about 250 mM, particularly an amount of about 230 mM or about 240 mM, most preferably in an amount of about 230 mM.

Prasinezumab (PRX002/RG7935/R07046015/NEOD002) is an immunoglobulin class G1 (IgG1) humanized monoclonal antibody (mAb) derived from murine parental antibody 9E4 (produced by the hybridoma given ATCC Accession Number PTA-8221) and is directed against an epitope in the C-terminus of human a synuclein (amino acids 118-126). Prasinezumab binds in biochemical and biophysical experiments to both soluble and insoluble forms of human α-synuclein, and with a greater relative affinity/avidity to aggregated over monomeric forms of α-synuclein. The amino acid sequence of Prasinezumab's heavy chain variable region (VH) is set forth in Seq. Id. No. 1 and the amino acid sequence of Prasinezumab's light chain variable region (VL) is set forth in Seq. Id. No. 2. Prasinezumba's heavy chain amino acid sequence is set forth in Seq. Id. No. 3 with or without the C-terminal lysine and the ligth chain amino acid sequence is set forth in Seq. Id. No. 4.

The term “amino acid” as used herein denotes a pharmaceutically acceptable organic molecule possessing an amino moiety located at α-position to a carboxylic group. Examples of amino acids include but are not limited to arginine, glycine, ornithine, lysine, histidine, glutamic acid, asparagic acid, isoleucine, leucine, alanine, phenylalanine, tyrosine, tryptophane, methionine, serine, proline. The amino acid employed is preferably in each case the L-form. Basic amino acids, such as arginine, histidine, or lysine, are preferably employed in the form of their inorganic salts (advantageously in the form of the hydrochloric acid salts, i.e. as amino acid hydrochlorides). A preferred amino acid for use in the present invention is methionine. Methionine is preferably used at a concentration of about 5 to about 25 mM, most preferably about 10 mM.

A subgroup within the stabilizers are antioxidants. The term “antioxidant” denotes pharmaceutically acceptable excipients, which prevent oxidation of the active pharmaceutical ingredient. Antioxidants comprise but are not limited to ascorbic acid, gluthathione, cysteine, methionine, citric acid, EDTA. Antioxidants can be used in an amount of about 0.01 to about 100 mM, preferably in an amount of about 5 to about 50 mM and more preferably in an amount of about 5 to about 25 mM.

The formulations according to the invention may also comprise one or more tonicity agents. The term “tonicity agents” denotes pharmaceutically acceptable excipients used to modulate the tonicity of the formulation. The formulation can be hypotonic, isotonic or hypertonic. Isotonicity in general relates to the osmotic pressure of a solution, usually relative to that of human blood serum (around 250-350 mOsmol/kg). The formulation according to the invention can be hypotonic, isotonic or hypertonic but will preferably be isotonic. An isotonic formulation is liquid or liquid reconstituted from a solid form, e.g. from a lyophilized form, and denotes a solution having the same tonicity as some other solution with which it is compared, such as physiologic salt solution and the blood serum. Suitable tonicity agents comprise but are not limited to sodium chloride, potassium chloride, glycerine and any component from the group of amino acids or sugars, in particular glucose. Tonicity agents are generally used in an amount of about 5 mM to about 500 mM.

Within the stabilizers and tonicity agents there is a group of compounds which can function in both ways, i.e. they can at the same time be a stabilizer and a tonicity agent. Examples thereof can be found in the group of sugars, amino acids, polyols, cyclodextrines, polyethyleneglycols and salts. An example for a sugar which can at the same time be a stabilizer and a tonicity agent is trehalose.

The formulations may also contain adjuvants such as preservatives, wetting agents, emulsifying agents and dispersing agents. Prevention of presence of microorganisms may be ensured both by sterilization procedures, and by the inclusion of various antibacterial and antifungal agents, e.g. paraben, chlorobutanol, phenol, sorbic acid, and the like. Preservatives are generally used in an amount of about 0.001 to about 2% (w/v). Preservatives comprise but are not limited to ethanol, benzyl alcohol, phenol, m-cresol, pchlor-m-cresol, methyl or propyl parabens, benzalkonium chloride.

As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

As used herein, the term “antigen binding moiety” refers to a polypeptide molecule that specifically binds to an antigenic determinant. In one embodiment, an antigen binding moiety is able to direct the entity to which it is attached (e.g. a second antigen binding moiety) to a target site, for example to a specific type of tumor cell bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antibody comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

The term “valent” as used herein denotes the presence of a specified number of antigen binding sites in an antibody. As such, the term “monovalent binding to an antigen” denotes the presence of one (and not more than one) antigen binding site specific for the antigen in the antibody.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′) 2, diabodies, linear antibodies, singlechain antibody molecules (e.g. scFv), and single-domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′) 2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., lgG₁, IgG₂, IgG₃, IgG₄, IgA₁, and IgA₂. The heavy chain constant domains that correspond to the different classes of immunoglobulins are called α, δ, ε, γ, and μ, respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

The formulations may also contain adjuvants such as preservatives, wetting agents, emulsifying agents and dispersing agents. Prevention of presence of microorganisms may be ensured both by sterilization procedures, and by the inclusion of various antibacterial and antifungal agents, e.g. paraben, chlorobutanol, phenol, sorbic acid, and the like. Preservatives are generally used in an amount of about 0.001 to about 2% (w/v). Preservatives comprise but are not limited to ethanol, benzyl alcohol, phenol, m-cresol, p-chlor-m-cresol, methyl or propyl parabens, benzalkonium chloride.

The pharmaceutical formulation may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol, sorbic acid, and the like. Preservatives are generally used in an amount of about 0.001 to about 2% (w/v). Preservatives comprise but are not limited to ethanol, benzyl alcohol, phenol, m-cresol, p-chlor-m-cresol, methyl or propyl parabens, benzalkonium chloride.

A formulation of the present invention can be administered by a variety of methods known in the art. As will be appreciated by the skilled artisan, the route and/or mode of administration will vary depending upon the desired results. To administer a formulation of the invention by certain routes of administration, it may be necessary to dilute the formulation in a diluent. Pharmaceutically acceptable diluents include saline, glucose, Ringer and aqueous buffer solutions.

Preferably, the formulation according to the invention is administered by intravenous (IV), subcutaneous (SC), or any other parental administration means such as those known in the pharmaceutical art. In a preferred aspect, the pharmaceutical formulation is administered by IV infusion. When administered via intravenous injection, it may be administered as a bolus injection or as a continuous infusion. For instance, the pharmaceutical formulation of the invention can be diluted with a sterile saline solution and administered with an infusion pump as normally used in clinical setting.

The phrases “parenteral administration” and “administered parenterally” as used herein mean modes of administration other than enteral and topical administration, usually by injection, and include, without limitation, intravenous, intramuscular, intra-arterial, intrathecal, extracapsular, infraorbital, intracardiac, intradermal, intraperitoneal, transtracheal, subcutaneous, subcuticular, intraarticular, subcapsular, subarachnoid, intraspinal, epidural and intrasternal injection and infusion.

The pharmaceutical formulation according to the invention is suitably administered to the patient at one time or over a series of treatments and may be administered to the patient at any time from diagnosis onwards; it may be administered as the sole treatment or in conjunction with other drugs or therapies useful in treating the conditions as described herein before.

The antibody against alpha synuclein may be the sole active ingredient in the liquid pharmaceutical composition. Alternatively, the antibody against alpha synuclein may be administered in combination, e.g. simultaneously, sequentially or separately, with one or more other therapeutically active ingredients. The term “active ingredient” as used herein refers to an ingredient with a pharmacological effect, such as a therapeutic effect, at a relevant dose.

The pharmaceutical compositions suitably comprise a therapeutically effective amount of antibody. The term “therapeutically effective amount” as used herein refers to an amount of a therapeutic agent needed to treat, ameliorate or prevent a targeted disease or condition, or to exhibit a detectable therapeutic, pharmacological or preventative effect. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy.

This amount can be determined by routine experimentation and is within the judgement of the clinician. In a particular aspect, the antibody against alpha synuclein is administered at a (fixed) dose of 600-1200 mg, particularly at a dose of 750-1100 mg, more particularly at a dose of 750-1000 mg and even more particularly at a dose of 900-1000 mg. In a preferred aspect, the dose is 1000 mg.

The stable formulations to be used for in vivo administration must be sterile. This is readily accomplished by filtration through sterile filtration membranes. The formulation must be fluid to the extent that the formulation is deliverable by syringe or an infusion system. In addition to water, the carrier can be an isotonic buffered saline solution, ethanol, polyol (e.g., glycerol, propylene glycol, and liquid polyethylene glycol, and the like), and suitable mixtures thereof. In view of their high stability the pharmaceutical formulation according to the invention can be administered i.v. without the need of an in-line filter and is thus much more convenient to handle than conventional formulations that need to be administered with an in-line filter. In-line filters such as Sterifix® have to be installed in the infusion line of i.v. medications to prevent the administration of any particles, air, or microorganisms that may be in the i.v. solution or line. Particles of 5 to 20 microns size and larger have the capability of obstructing blood flow through pulmonary capillaries, which could lead to complications such as pulmonary embolism. Foreign particles can also cause phlebitis at the injection site and filters may help to reduce the incidence of phlebitis.

The stable pharmaceutical formulation according to the invention can be prepared by methods known in the art, e.g. ultrafiltration-diafiltration, dialysis, addition and mixing, lyophilisation, reconstitution, and combinations thereof. Examples of preparations of formulations according to the invention can be found herein after.

The invention thus comprises a process for the preparation of the formulations according to the invention. Said process comprises buffer-exchanging the antibody against a diafiltration buffer containing the anticipated buffer composition, and, where required, concentration of the antibody by diafiltration, followed by adding the excipients (e.g., sucrose, sodium chloride, methionine) as stock solutions to the antibody solution, followed by adding the surfactant as stock solution to the antibody/excipient solution, and finally adjusting the antibody concentration to the desired final concentration using buffer solution, whereby also the final excipient and surfactant concentrations are reached.

Alternatively, the excipients can also be added as solids to the starting solution comprising the antibody. If the antibody is in the form of a solid, e.g. a lyophilizate, the formulation according to the invention can be prepared by firstly dissolving the antibody in water or buffer solution, optionally comprising one or more of the excipients, and subsequently adding the further excipients as stock solutions or solids. The antibody can advantageously also be dissolved directly in a solution comprising all further excipients. One or more of the excipients present in the formulation according to the invention may already be added during or at the end of the process for the preparation of the antibody, e.g. by dissolving the antibody directly in a solution comprising one, more than one, or preferably all of the excipients of the formulation in the final step of the purification carried out after the preparation of the antibody. If the solution comprising the antibody and the excipients does not yet have the desired pH, this is adjusted by addition of an acid or base, preferably using the acid or base already present in the buffer system. This is followed by sterile filtration.

The stable liquid pharmaceutical formulations according to the invention can also be in a lyophilized form or in a liquid form reconstituted from the lyophilized form. The “lyophilized form” is manufactured by freeze-drying methods known in the art. The lyophilizate usually has a residual moisture content of about 0.1 to 5% (w/w) and is present as a powder or a physically stable cake. The “reconstituted form” can be obtained from the lyophilizate by a fast dissolution after addition of reconstitution medium. Suitable reconstitution media comprise but are not limited to water for injection (WFI), bacteriostatic water for injection (BWFI), sodium chloride solutions (e.g. 0.9% (w/v) NaCl), glucose solutions (e.g. 5% (w/v) glucose), surfactant-containing solutions (e.g. 0.01% (w/v) polysorbate 20 and pH-buffered solutions (e.g. phosphate-buffered solutions).

In another preferred embodiment, the buffering agent comprised in the formulation according to the invention is a histidine buffer, preferably an L-histidine/HCl buffer. Particularly preferred is an L-histidine/acetate buffer (i.e. L-histidine as the buffering agent).

Preferably, the buffering agent provides a pH of 5.0 to 6.0, more preferably 5.5±0.5, most preferably 5.5±0.3.

In a preferred embodiment, the surfactant comprised in the formulation according to the invention is a polysorbate, preferably polysorbate 20 or polysorbate 80, most preferably polysorbate 20.

Preferably, the surfactant is at a concentration of 0.01 to 0.1% (w/v), more preferably 0.02 to 0.05%, most preferably 0.04%.

Preferably, the at least one stabilizer is at a concentration of 120 to 300 mM, more preferably 220 to 250 mM, most preferably 230 to 240 mM.

The formulation according to the invention can be in a liquid form, in a lyophilized form or in a liquid form reconstituted from a lyophilized form. In certain embodiments, the formulation is in a liquid form.

The term “liquid” as used herein in connection with the formulation according to the invention denotes a formulation which is liquid at a temperature of at least about 2 to about 8° C. under atmospheric pressure.

The term “lyophilized” as used herein in connection with the formulation according to the invention denotes a formulation which is manufactured by freeze-drying methods known in the art per se. The solvent (e.g. water) is removed by freezing followed by sublimation of the ice under vacuum and desorption of residual water at elevated temperature. The lyophilizate usually has a residual moisture of about 0.1 to 5% (w/w) and is present as a powder or a physically stable cake. The lyophilizate is characterized by a fast dissolution after addition of a reconstitution medium.

The term “reconstituted form” as used herein in connection with the formulation according to the invention denotes a formulation which is lyophilized and re-dissolved by addition of reconstitution medium. Suitable reconstitution media comprise but are not limited to water for injection (WFI), bacteriostatic water for injection (BWFI), sodium chloride solutions (e.g. 0.9% (w/v) NaCl), glucose solutions (e.g. 5% glucose), surfactant-containing solutions (e.g. 0.01% polysorbate 20), pH-buffered solutions (e.g. phosphate-buffered solutions).

The formulation according to the invention is physiologically well tolerated, can be prepared easily, can be dispensed precisely and is stable with respect to decomposition products and aggregates over the duration of storage, during repeated freezing and thawing cycles and mechanical stress.

The invention further comprises a process for the preparation of the formulations according to the invention. Said process comprises buffer-exchanging the alpha synuclein antibody against a diafiltration buffer containing the anticipated buffer composition, and, where required, concentration of the antibody by diafiltration, followed by adding the excipients (e.g. trehalose dihydrate, sucrose, arginine, sodium chloride, methionine) as stock solutions to the antibody solution, followed by adding the surfactant as stock solution to the antibody/excipient solution, and finally adjusting the antibody concentration to the desired final concentration using buffer solution, whereby also the final excipient and surfactant concentrations are reached.

Alternatively, the excipients can also be added as solids to the starting solution comprising the alpha synuclein antibody. If the alpha synuclein antibody is in the form of a solid, e.g. a lyophilizate, the formulation according to the invention can be prepared by firstly dissolving the bispecific antibody in water or buffer solution, optionally comprising one or more of the excipients, and subsequently adding the further excipients as stock solutions or solids. The alpha synuclein antibody can advantageously also be dissolved directly in a solution comprising all further excipients. One or more of the excipients present in the formulation according to the invention may already be added during or at the end of the process for the preparation of the alpha synuclein antibody, e.g. by dissolving the alpha synuclein antibody directly in a solution comprising one, more than one, or preferably all of the excipients of the formulation in the final step of the purification carried out after the preparation of the alpha synuclein antibody. If the solution comprising the alpha synuclein antibody and the excipients does not yet have the desired pH, this is adjusted by addition of an acid or base, preferably using the acid or base already present in the buffer system. This is followed by sterile filtration.

The invention further comprises the formulations according to the invention for use in treating diseases, or the use of the formulations according to the invention for the preparation of a medicament useful for treating diseases, particularly cell proliferation disorders, wherein CEA is expressed, particularly wherein CEA is abnormally expressed (e.g., overexpressed) compared to normal tissue of the same cell type. Such disorders include different types of cancer, such as colorectal cancer, lung cancer, pancreatic cancer, breast cancer, and gastric cancer. CEA expression levels may be determined by methods known in the art (e.g., via immunohistochemistry assay, immunofluorescence assay, immunoenzyme assay, ELISA, flow cytometry, radioimmunoassay, Western blot, ligand binding, kinase activity, etc.). The invention also comprises methods for treating diseases as described hereinabove, comprising administering a formulation according to the invention to an individual in need thereof.

To administer a formulation of the invention by certain routes of administration, it may be necessary to dilute the formulation in a diluent. Pharmaceutically acceptable diluents include saline, glucose, Ringer and aqueous buffer solutions.

Preferably, the formulation according to the invention is administered by intravenous (i.v.), subcutaneous (s.c.), or any other parental administration means such as those known in the pharmaceutical art.

The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The formulation according to the invention can be prepared by methods known in the art, e.g. ultrafiltration-diafiltration, dialysis, addition and mixing, lyophilisation, reconstitution, and combinations thereof. Examples of preparations of formulations according to the invention can be found hereinafter.

Prasinezumab amino acid sequences

| Seq. | | |
|---|---|---|
| Id. | | |
| No. | Name | Sequence |
| 1 | Variable | EVQLVESGGGLVQPGGSLRLSCAASGFTFSNYGMSWVRQAPGKGLE |
| | heavy chain | WVASISSGGGSTYYPDNVKGRFTISRDDAKNSLYLQMNSLRAEDTAVY |
| | VH | YCARGGAGIDYWGQGTLVTVSS |
| 2 | Variable | DIQMTQSPSSLSASVGDRVTITCKSIQTLLYSSNQKNYLAWFQQKPGK |
| | light chain | APKLLIYWASIRKSGVPSRFSGSGSGTDFTLTISSLQPEDLATYYCQQYY |
| | VL | SYPLTFGGGTKLEIK |
| 3 | Heavy chain | EVQLVESGGGLVQPGGSLRLSCAASGFTFSNYGMSWVRQAPGKG |
| | | LEWVASISSGGGSTYYPDNVKGRFTISRDDAKNSLYLQMNSLRAED |
| | | TAVYYCARGGAGIDYWGQGTLVTVSSASTKGPSVFPLAPSSKSTSGG |
| | | TAALGCLVKDYFPEPVTVSWNSGALTSGVHTFPAVLQSSGLYSLSS |
| | | VVTVPSSSLGTQTYICNVNHKPSNTKVDKRVEPKSCDKTHTCPPCPAP |
| | | ELLGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPSDI |
| | | AVEWESNGQPENNYKTTPPVLDSDGSFFLYSKLTVDKSRWQQGNVF |
| | | SCSVMHEALHNHYTQKSLSLSPGK |
| 4 | Light chain | DIQMTQSPSSLSASVGDRVTITCKSIQTLLYSSNQKNYLAWFQQK |
| | | PGKAPKLLIYWASIRKSGVPSRFSGSGSGTDFTLTISSLQPEDLA |
| | | TYYCQQYYSYPLTFGGGTKLEIKRTVAAPSVFIFPPSDEQLKSGTASVVC |
| | | LLNNFYPREAKVQWKVDNALQSGNSQESVTEQDSKDSTYSLSSTLT- |
| | | LSKADYEKHKVYACEVTHQGLSSPVTKSFNRGEC |

The examples explain the invention in more detail but should not be construed as limiting the scope of the invention. The disclosures of all patent and scientific literature cited herein are expressly incorporated in their entirety by reference.

### EXAMPLES

The anti-synuclein alpha antibody formulations according to the invention were developed based on the experimental results as provided below using the general preparatory and analytical methods and assays as outlined below.

Liquid drug product formulations according to the invention were developed as follows.

### Example 1; Preparation of the Liquid Formulations

For the preparation of the liquid formulations prasinezumab was buffer-exchanged against a diafiltration buffer containing the anticipated buffer composition and where required, concentrated by diafiltration to an antibody concentration approx. 20-30% above target concentration. After completion of the diafiltration operation, the excipients (e.g. sucrose, sodium chloride, and methionine) were added as stock solutions to the antibody solution. The surfactant was then added as a 50 to 200-fold stock solution. Finally, the protein concentration was adjusted with a buffer to the final prasinezumab concentration of approx. 150 mg/ml.

All formulations were sterile-filtered through 0.22 μm low protein binding filters and aseptically filled into sterile 6 ml glass vials closed with ETFE (copolymer of ethylene and tetrafluoroethylene)-coated rubber stoppers and aluminum crimp caps. The fill volume was approx. 2.7 ml. These formulations were stored at different ICH climate conditions (5° C., 25° C. and 40° C.) for different intervals of time and stressed by shaking (1 week at a shaking frequency of 200 min-1 at 5° C. and 25° C.) and freeze-thaw stress methods. The samples were analyzed before and after applying the stress tests by the following analytical methods:

The samples were analyzed before and after applying the stress tests by the following analytical methods
UV spectrophotometry
Size Exclusion Chromatography (SEC)
Imaged capillary electrophoresis/capillary isoelectric focusing (iCE/cIEF) Ion exchange chromatography (IEC)
Clarity and opalescence of the solution.
Analytical Protein A Chromatography.
Visual inspection

UV spectroscopy, used for determination of protein content, was performed on a Perkin Elmer 235 UV spectrophotometer in a wavelength range from 240 nm to 400 nm. Neat protein samples were diluted to approx. 0.5 mg/mL with the corresponding formulation buffer. The protein concentration was calculated according to equation 1.

$$Protein content = \frac{A ( 280 ) - A ( 320 ) \times dil . factor}{ε 〈 {cm}^{2} / mg 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient s.

Size Exclusion Chromatography (SEC) was used to detect soluble high molecular weight species (aggregates) and low molecular weight hydrolysis products (LMW) in the formulations. The method was performed on a Waters Alliance 2695 HPLC instrument with a Waters W2487 Dual Absorbance Detector and equipped with a Waters BioSuite 250 column. Intact monomer, aggregates and hydrolysis products were separated by an isocratic elution profile, using 0.2M K2HPO4/0.25M KCL, pH 7.0 as mobile phase, and were detected at a wavelength of 280 nm.

Imaged capillary electrophoresis/cappilary isoelectric focusing (iCE/cIEF) was used to characterize the different charge species of the antibody prasinezumab in the formulation. The equipment used for formulation F1 is ICE3 IEF Analyzer from Convergent Bioscience (Protein Simple). The equipment used for F4n, F5n, F8n, and F9n is iCE280 IEF Analyzer from the same manufacturer. Cartridge FC-coated capillary (code 101701). Pre-focusing: 1 Min/1500 V (all). Focusing: 11 Min/3000 V all without Hb Std (4.5 Min/3000 V). Detection wavelength: 280 nm. Low pI marker: 8.18/0.01 mg/ml. High pI marker: 9.77/0.01 mg/ml. Pharmalyte: 8-10.5/3%. Biolyte: 7-9/1%. Sample pre-dilution: 2.5 mg/ml with Mili-Q water. Centrifugation: before loading 10000 rpm. Sample temperature: 10° C., Standard: Hemoglobin stock solution, Bioscience, Art. Nr. 101801. Sample concentration: 0.25 mg/ml.

Clarity and the degree of opalescence were measured as Formazine Turbidity Units (FTU) by the method of nephelometry. The neat sample was transferred into a 11 mm diameter clear-glass tube and placed into a HACH 2100AN turbidimeter.

Analytical Protein A chromatography was performed to monitor the oxidation status of the four conserved methionine side chains in the Fc part of the alpha synuclein antibody. The method was performed on a Waters Alliance 2695 HPLC instrument with a Waters W2487 Dual Absorbance Detector (detection wavelength 280 nm), equipped with a Poros A720 4.6 mm×50 mm column from Applied Biosystems, USA. PBS from Gibco, Invitrogen and 0.1M acetic acid, 0.15M sodium chloride, pH 2.8 were used as mobile phases A and B, respectively, at a flow rate of 2.0 mL/min:

Samples were inspected for the presence of visible particles by using a Simplex Ampoule Testing Apparatus OPTIMA I (European Pharmacopoeia (EP) box method).

The results of the stability testing for the Formulation F4n to F9n and F1 are provided in the tables below. Formulation F1 was determined to be most favorable for obtaining maximum antibody concentrations, maximum antibody stability and antibody formulations free from particle

F9n is a liquid formulation with the composition 150 mg/mL prasinezumab, 20 mM Histidine
Acetate, 130 mM Arginine-HCl, 10 mM Methionine, 0.04% Polysorbate 20 at pH 5.5

| | | | | | iCE/cIEF | | | | |
|---|---|---|---|---|---|---|---|---|---|
| | Storage | Size Exclusion-HPLC | | | Main | Acidic | Basic | | |
| Storage | Time | Monomer | HMW | LMW | Peak | Species | species | Turbidity | Visible particles |
| condition | (week) | (%) | (%) | (%) | (%) | (%) | (%) | (FTU) | (EP box method) |
| — | Initial | 98.4 | 1.5 | 0.1 | 42.2 | 51.1 | 6.6 | 20.2 | Practically free from |
| | | | | | | | | | Particles |
| 5° C. | 4 | 98.1 | 1.7 | 0.1 | 44.3 | 50.3 | 5.4 | 19.9 | Practically free from |
| | | | | | | | | | Particles |
| 5° C. | 8 | 98.1 | 1.8 | 0.2 | 36.9 | 56.9 | 6.2 | 20.3 | Practically free from |
| | | | | | | | | | Particles |
| 5° C. | 12 | 98 | 1.9 | 0.1 | 40.2 | 54.6 | 5.2 | 19.8 | Practically free from |
| | | | | | | | | | Particles |
| 5° C. | 26 | 97.9 | 2 | 0.1 | 42.6 | 51.8 | 5.6 | 19.6 | Practically free from |
| | | | | | | | | | Particles |
| 5° C. | 39 | 97.9 | 2.1 | 0.1 | 38.3 | 56.2 | 5.5 | 19.7 | Practically free from |
| | | | | | | | | | Particles |
| 5° C. | 52 | 97.8 | 2.1 | 0.1 | 42.7 | 52.7 | 4.6 | 19.1 | Data not available |
| 25° C. | 4 | 97.8 | 2 | 0.2 | 42.6 | 51.5 | 5.8 | 19.5 | Practically free from |
| | | | | | | | | | Particles |
| 25° C. | 8 | 97.5 | 2.2 | 0.3 | 36.5 | 57.5 | 6 | 18.6 | Practically free from |
| | | | | | | | | | Particles |
| 25° C. | 12 | 97.4 | 2.3 | 0.2 | 38.3 | 55.8 | 5.9 | 18.8 | Practically free from |
| | | | | | | | | | Particles |
| 25° C. | 26 | 97.1 | 2.5 | 0.5 | 36.1 | 57.7 | 6.2 | 18.2 | Practically free from |
| | | | | | | | | | Particles |
| 25° C. | 39 | 96.7 | 2.7 | 0.6 | 31.9 | 61.9 | 6.2 | 17.8 | Practically free from |
| | | | | | | | | | Particles |
| 25° C. | 52 | 96.4 | 2.8 | 0.8 | 31.3 | 62.6 | 6 | 17.3 | Data not available |
| 40° C. | 4 | 96.6 | 2.7 | 0.7 | 29.5 | 63.6 | 6.9 | 18.6 | Practically free from |
| | | | | | | | | | Particles |
| 40° C. | 8 | 95.6 | 3.3 | 1.1 | 23.6 | 69.7 | 6.7 | 17.7 | Practically free from |
| | | | | | | | | | Particles |
| 40° C. | 12 | 94.4 | 4 | 1.6 | 15.9 | 78.1 | 6 | 18.1 | Practically free from |
| | | | | | | | | | Particles |
| 40° C. | 26 | 91.4 | 5.7 | 2.9 | 9.6 | 84.4 | 6 | 17.9 | Practically free from |
| | | | | | | | | | Particles |
| 5 Freeze | 1 | 98.2 | 1.7 | 0.1 | 43.4 | 51.3 | 5.2 | 20.4 | Practically free from |
| Thaw cycles | | | | | | | | | Particles |
| 5° C. | 1 | 98.2 | 1.7 | 0.1 | 44.2 | 51.1 | 4.7 | 19.9 | Practically free from |
| Shaking | | | | | | | | | Particles |

F8n is a liquid formulation with the composition 150 mg/mL prasinezumab, 20 mM Histidine
Acetate, 130 mM Arginine-HCl, 10 mM Methionine, 0.06% (w/v) Poloxamer 188 at pH 5.5

| | | | | | iCE/cIEF | | | | |
|---|---|---|---|---|---|---|---|---|---|
| | Storage | Size Exclusion-HPLC | | | Main | Acidic | Basic | | |
| Storage | Time | Monomer | HMW | LMW | Peak | Species | species | Turbidity | Visible particles |
| condition | (week) | (%) | (%) | (%) | (%) | (%) | (%) | (FTU) | (EP box method) |
| — | Initial | 98.4 | 1.5 | 0.1 | 42.9 | 52 | 5.1 | 23.4 | Practically free from |
| | | | | | | | | | Particles |
| 5° C. | 4 | 98.2 | 1.7 | 0.1 | 45.7 | 49.4 | 5 | 23.7 | Practically free from |
| | | | | | | | | | Particles |
| 5° C. | 8 | 98.1 | 1.8 | 0.2 | 37 | 56.9 | 6.1 | 23.5 | Practically free from |
| | | | | | | | | | Particles |
| 5° C. | 12 | 98 | 1.9 | 0.1 | 41 | 54 | 5 | 23.3 | Practically free from |
| | | | | | | | | | Particles |
| 5° C. | 26 | 97.9 | 2 | 0.1 | 42.5 | 52 | 5.5 | 23 | Practically free from |
| | | | | | | | | | Particles |
| 5° C. | 39 | 97.9 | 2.1 | 0.1 | 37.8 | 56.4 | 5.8 | 22.6 | Practically free from |
| | | | | | | | | | Particles |
| 5° C. | 52 | 97.8 | 2.1 | 0.1 | 42.1 | 52.7 | 5.2 | 23.1 | Data not available |
| 25° C. | 4 | 97.8 | 2 | 0.2 | 43.2 | 51.7 | 5.1 | 22.9 | Practically free from |
| | | | | | | | | | Particles |
| 25° C. | 8 | 97.6 | 2.2 | 0.3 | 36.4 | 57.6 | 6 | 22.6 | Practically free from |
| | | | | | | | | | Particles |
| 25° C. | 12 | 97.4 | 2.4 | 0.3 | 38.4 | 56.2 | 5.5 | 22.9 | Practically free from |
| | | | | | | | | | Particles |
| 25° C. | 26 | 97.1 | 2.5 | 0.4 | 35.8 | 57.9 | 6.3 | 21.7 | Practically free from |
| | | | | | | | | | Particles |
| 25° C. | 39 | 96.7 | 2.7 | 0.6 | 31.5 | 61.9 | 6.6 | 23.8 | Practically free from |
| | | | | | | | | | Particles |
| 25° C. | 52 | 96.3 | 2.9 | 0.8 | 30.8 | 62.6 | 6.6 | 21.3 | Data not available |
| 40° C. | 4 | 96.6 | 2.7 | 0.7 | 29.9 | 62.6 | 7.5 | 21.3 | Practically free from |
| | | | | | | | | | Particles |
| 40° C. | 8 | 95.5 | 3.3 | 1.1 | 22.9 | 69.1 | 8.1 | 21 | Practically free from |
| | | | | | | | | | Particles |
| 40° C. | 12 | 94.3 | 4.1 | 1.6 | 16.5 | 77.4 | 6.1 | 20.9 | Practically free from |
| | | | | | | | | | Particles |
| 40° C. | 26 | 91.4 | 5.7 | 3 | 9.9 | 83.5 | 6.7 | 20.3 | Practically free from |
| | | | | | | | | | Particles |
| 5 Freeze | 1 | 98.2 | 1.7 | 0.1 | 42.9 | 51.7 | 5.3 | 23.5 | Practically free from |
| Thaw | | | | | | | | | Particles |
| cycles | | | | | | | | | |
| 5° C. | 1 | 98.2 | 1.7 | 0.1 | 41.5 | 52.9 | 5.7 | 23.2 | Practically free from |
| Shaking | | | | | | | | | Particles |
| 25° C. | 1 | 98 | 1.8 | 0.2 | 44.1 | 51.2 | 4.7 | 23 | Practically free from |
| Shaking | | | | | | | | | Particles |

F5n is a liquid formulation with the composition 150 mg/mL prasinezumab, 20 mM Histidine
Acetate, 220 mM Sucrose, 10 mM Methionine, 0.04% (w/v) Polysorbate 20 at pH 5.5

| | | | | | iCE/cIEF | | | | |
|---|---|---|---|---|---|---|---|---|---|
| | Storage | Size Exclusion-HPLC | | | Main | Acidic | Basic | Turbid- | |
| Storage | Time | Monomer | HMW | LMW | Peak | Species | species | ity | Visible particles |
| condition | (week) | (%) | (%) | (%) | (%) | (%) | (%) | (FTU) | (EP box method) |
| — | Initial | 98.3 | 1.6 | 0.1 | 37.9 | 57.5 | 4.7 | 4.13 | Practically free from |
| | | | | | | | | | Particles |
| 5° C. | 4 | 98.1 | 1.8 | 0.1 | 40.7 | 53.7 | 5.7 | 4.06 | Practically free from |
| | | | | | | | | | Particles |
| 5° C. | 8 | 98.1 | 1.8 | 0.1 | 41 | 52.5 | 6.5 | 3.86 | Practically free from |
| | | | | | | | | | Particles |
| 5° C. | 12 | 97.9 | 2 | 0.1 | 42.6 | 51.9 | 5.5 | 3.87 | Practically free from |
| | | | | | | | | | Particles |
| 5° C. | 30 | 97.7 | 2.2 | 0.1 | 42.4 | 52.2 | 5.5 | 4.17 | Practically free from |
| | | | | | | | | | Particles |
| 25° C. | 4 | 97.6 | 2.3 | 0.1 | 41.4 | 53.9 | 4.7 | 4.16 | Practically free from |
| | | | | | | | | | Particles |
| 25° C. | 8 | 97.3 | 2.5 | 0.2 | 37.2 | 55.9 | 6.9 | 3.98 | Practically free from |
| | | | | | | | | | Particles |
| 25° C. | 12 | 97 | 2.7 | 0.3 | 36.5 | 57.6 | 5.9 | 3.95 | Practically free from |
| | | | | | | | | | Particles |
| 25° C. | 30 | 96.2 | 3.3 | 0.5 | 30.4 | 63.2 | 6.5 | 4.2 | Practically free from |
| | | | | | | | | | Particles |
| 40° C. | 4 | 96 | 3.4 | 0.6 | 25.4 | 68.6 | 6 | 4.31 | Practically free from |
| | | | | | | | | | Particles |
| 40° C. | 8 | 94.7 | 4.3 | 1 | 17.5 | 75.7 | 6.8 | 4.27 | Practically free from |
| | | | | | | | | | Particles |
| 40° C. | 12 | 93.4 | 5.2 | 1.5 | 13.1 | 81.3 | 5.6 | 4.4 | Practically free from |
| | | | | | | | | | Particles |
| 40° C. | 30 | 85 | 11.7 | 3.3 | 4.9 | 92.8 | 2.3 | 5.84 | Practically free from |
| | | | | | | | | | Particles |
| 5 Freeze | 1 | 98.1 | 1.8 | 0.1 | 41.1 | 53.6 | 5.4 | 4.03 | Practically free from |
| Thaw | | | | | | | | | Particles |
| cycles | | | | | | | | | |
| 5° C. | 1 | 98 | 1.9 | 0.1 | 39.1 | 54.6 | 6.3 | 3.97 | Practically free from |
| Shaking | | | | | | | | | Particles |
| 25° C. | 1 | 97.9 | 1.9 | 0.1 | 37.7 | 57.3 | 5 | 3.94 | Practically free from |
| Shaking | | | | | | | | | Particles |

F4n is a liquid formulation with the composition 150 mg/mL prasinezumab, 20 mM Histidine
Acetate, 220 mM Sucrose, 10 mM Methionine, 0.02% (w/v) Poloxamer 188 at pH 5.5

| | | | | | iCE/cIEF | | | | |
|---|---|---|---|---|---|---|---|---|---|
| | Storage | Size Exclusion-HPLC | | | Main | Acidic | Basic | Turbid- | |
| Storage | Time | Monomer | HMW | LMW | Peak | Species | species | ity | Visible particles |
| condition | (week) | (%) | (%) | (%) | (%) | (%) | (%) | (FTU) | (EP box method) |
| — | Initial | 98.3 | 1.6 | 0.1 | 37.4 | 58.1 | 4.4 | 4.45 | Practically free from |
| | | | | | | | | | Particles |
| 5° C. | 4 | 98.1 | 1.8 | 0.1 | 39.8 | 55 | 5.2 | 4.09 | Practically free from |
| | | | | | | | | | Particles |
| 5° C. | 8 | 98 | 1.9 | 0.1 | 40.3 | 53.5 | 6.3 | 3.81 | Practically free from |
| | | | | | | | | | Particles |
| 5° C. | 12 | 97.9 | 2 | 0.1 | 43.1 | 51.5 | 5.3 | 3.89 | Practically free from |
| | | | | | | | | | Particles |
| 5° C. | 30 | 97.7 | 2.2 | 0.1 | 41.9 | 52.2 | 5.9 | 4.12 | Practically free from |
| | | | | | | | | | Particles |
| 25° C. | 4 | 97.6 | 2.3 | 0.2 | 41.1 | 54.3 | 4.6 | 4.3 | Practically free from |
| | | | | | | | | | Particles |
| 25° C. | 8 | 97.3 | 2.5 | 0.2 | 37.9 | 55.4 | 6.8 | 3.93 | Practically free from |
| | | | | | | | | | Particles |
| 25° C. | 12 | 97 | 2.7 | 0.3 | 37.4 | 57.1 | 5.5 | 3.98 | Practically free from |
| | | | | | | | | | Particles |
| 25° C. | 30 | 96.2 | 3.3 | 0.5 | 30.2 | 62.5 | 7.3 | 4.4 | Practically free from |
| | | | | | | | | | Particles |
| 40° C. | 4 | 95.9 | 3.5 | 0.6 | 27 | 67.7 | 5.3 | 4.28 | Practically free from |
| | | | | | | | | | Particles |
| 40° C. | 8 | 94.7 | 4.3 | 1 | 17.4 | 75.9 | 6.7 | 4.35 | Practically free from |
| | | | | | | | | | Particles |
| 40° C. | 12 | 93.3 | 5.2 | 1.5 | 12.8 | 81.6 | 5.6 | 4.49 | Practically free from |
| | | | | | | | | | Particles |
| 40° C. | 30 | 85.1 | 11.7 | 3.2 | 4.2 | 91 | 4.8 | 5.63 | Practically free from |
| | | | | | | | | | Particles |
| 5 Freeze | 1 | 98.1 | 1.8 | 0.1 | 39 | 55 | 6 | 4.19 | Practically free from |
| Thaw | | | | | | | | | Particles |
| cycles | | | | | | | | | |
| 5° C. | 1 | 98 | 1.9 | 0.1 | 39.7 | 55.6 | 4.7 | 3.85 | Practically free from |
| Shaking | | | | | | | | | Particles |
| 25° C. | 1 | 97.9 | 2 | 0.1 | 37.8 | 57 | 5.2 | 3.99 | Practically free from |
| Shaking | | | | | | | | | Particles |

F1 is a liquid formulation with the composition 180 mg/mL prasinezumab, 20 mM Histidine
Acetate, 130 mM Arginine-HCl, 10 mM Methionine, 0.04% (w/v) Polysorbate 20 at pH 5.5

| | Storage | | | | iCE/cIEF | | | | |
|---|---|---|---|---|---|---|---|---|---|
| | Time | Size Exclusion-HPLC | | | Main | Acidic | Basic | | |
| Storage | (month | Monomer | HMW | LMW | Peak | Species | species | Turbidity | Visible particles |
| condition | or week) | (%) | (%) | (%) | (%) | (%) | (%) | (FTU) | (EP box method) |
| — | Initial | 99.2 | 0.8 | 0.1 | 40.7 | 46.5 | 12.8 | 12.8 | Practically free from |
| | | | | | | | | | Particles |
| 5° C. | 1 | 99.1 | 0.9 | 0.9 | 41.8 | 45.0 | 13.2 | 13.0 | Free from Particles |
| 5° C. | 3 | 99.0 | 0.9 | 0.1 | 36.4 | 52.1 | 11.6 | 13.1 | Free from Particles |
| 5° C. | 6 | 98.9 | 1.0 | 0.1 | 43.0 | 43.5 | 13.5 | 12.8 | Free from Particles |
| 5° C. | 9 | 98.9 | 1.0 | 0.1 | 37.7 | 53.0 | 9.3 | 12.2 | Free from Particles |
| 5° C. | 12 | 98.8 | 1.0 | 0.1 | 41. | 46.2 | 12.8 | 12.6 | Practically free from |
| | | | | | | | | | Particles |
| 5° C. | 24 | 98.7 | 1.1 | 0.1 | 40.8 | 45.0 | 14.2 | 12.3 | Practically free from |
| | | | | | | | | | Particles |
| 5° C. | 36 | 98.6 | 1.2 | 0.2 | 40.9 | 46 | 13.1 | 13.0 | Practically free from |
| | | | | | | | | | Particles |
| 25° C. | | 98.8 | 1.0 | 0.1 | 42.6 | 44.1 | 13.4 | 12.7 | Free from Particles |
| 25° C. | 3 | 98.5 | 1.2 | 0.3 | 35.5 | 54.4 | 10.1 | 12.7 | Free from Particles |
| 25° C. | 6 | 98.1 | 1.4 | 0.5 | 37.7 | 49.7 | 12.6 | 12.8 | Free from Particles |
| 25° C. | 12 | 97.4 | 1.8 | 0.9 | 35.0 | 51.4 | 13.6 | 12.6 | Free from Particles |
| 40° C. | 1 week | 98.7 | 1.1 | 0.2 | 36.9 | 46.1 | 15.0 | 12.6 | Free from Particles |
| 40° C. | 2 weeks | 98.3 | 1.3 | 0.4 | 33.4 | 51.2 | 15.5 | 12.9 | Free from Particles |
| 40° C. | 4 weeks | 97.7 | 1.7 | 0.6 | 31.5 | 53.2 | 15.3 | 12.1 | Free from Particles |
| 40° C. | 6 weeks | 97.2 | 2.0 | 0.9 | 27.5 | 62.8 | 9.8 | 12.8 | Free from Particles |
| 40° C. | 8 weeks | 96.6 | 2.3 | 1.1 | 24.1 | 67.0 | 8.9 | 12.7 | Free from Particles |

1. A pharmaceutical formulation comprising:
120 to 200 mg/ml of an alpha synuclein antibody;
1 to 50 mM of a buffering agent;
0.01 to 1% (w/v) of a surfactant;
1 to 50 mM of at least one stabilizer;
100-150 mM tonicity agent,
at a pH in the range of from 5.0 to 7.0.

2. The pharmaceutical formulation according to claim 1, wherein the anti-synuclein antibody concentration is in the range of 150 to 200 mg/ml.

3. The pharmaceutical formulation according to claim 1, wherein the buffering agent is a histidine buffer.

4. The pharmaceutical formulation according to claim 1, wherein the buffering agent is at a concentration of 10 to 40 mM.

5. The pharmaceutical formulation according to claim 1, wherein the buffering agent provides a pH of 5.0 to 6.0.

6. The pharmaceutical formulation according to claim 1, wherein the surfactant is a polysorbate.

7. The pharmaceutical formulation according to claim 1, wherein the surfactant is at a concentration of 0.01 to 0.1% (w/v).

8. The pharmaceutical formulation according to claim 1, wherein at least one stabilizer is selected from the group consisting of salts, saccharides, and amino acids.

9. The pharmaceutical formulation according to claim 1, wherein the at least one stabilizer is at a concentration of 5 to 50 mM.

10. The pharmaceutical formulation claim 1, wherein the tonicity agent is at a concentration of 120-150 mM.

11. The pharmaceutical formulation according to claim 10, wherein the tonicity agent is Arginine-HCl.

12. The pharmaceutical formulation according to claim 1, which comprises:
150 to 180 mg/ml alpha synuclein antibody;
15 to 30 mM L-histidine/acetate buffer;
0.02 to 0.05% (w/v) polysorbate 20;
120 to 180 mM arginine-HCl;
5 to 25 mM L-methionine;
at a pH of 5.5±0.5.

13. The pharmaceutical formulation according to claim 1, which comprises:
180 mg/ml alpha synuclein antibody,
20 mM L-histidine/acetate buffer,
0.04% (w/v) polysorbate 20,
130 mM arginine-HCl,
10 mM methionine, at a pH of 5.5.

14. The pharmaceutical formulation according to claim 1, wherein the anti-alpha synuclein antibody comprises a VH domain having the amino acid sequence of Seq. Id. No. 1; and VL domain having the amino acid sequence of Seq. Id. No. 2.

15. The pharmaceutical formulation according to claim 1, wherein the alpha synuclein antibody comprises a heavy chain having the amino acid sequence of Seq. Id. No. 3 and a light chain having amino acid sequence of Seq. Id. No. 4.

16. The pharmaceutical formulation according to claim 1, wherein the alpha synuclein antibody is the antibody with the INN prasinezumab.

17. The pharmaceutical formulation according to claim 1, which is in a liquid form, in a lyophilized form or in a liquid form reconstituted from a lyophilized form.

18. The pharmaceutical formulation according to claim 16, wherein the formulation is a liquid formulation.

19. The pharmaceutical formulation according to claim 1, wherein the anti-synuclein antibody is 180 mg/ml.

20. The pharmaceutical formulation according to claim 1, wherein the buffering agent is a histidine acetate buffer.

21. The pharmaceutical formulation according to claim 1, wherein the buffering agent provides a pH of 5.5.

22. The pharmaceutical formulation according to claim 6, wherein the surfactant is a polysorbate 20.

23. The pharmaceutical formulation according to claim 8, wherein the stabilizer is methionine.

24. The pharmaceutical formulation according to claim 10, wherein the tonicity agent is at a concentration of 130 mM.

25. The pharmaceutical formulation according to claim 18, wherein the liquid formation is for subcutaneous administration.

26. The pharmaceutical formulation according to claim 18, wherein the liquid formation is for i.v. administration.


SEQUENCE LISTING (4 sequences)

SEQ ID NO: 1 - protein, 116 aa, synthetic construct
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSS

SEQ ID NO: 2 - protein, 113 aa, synthetic construct
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIK

SEQ ID NO: 3 - protein, 446 aa, synthetic construct
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 220 aa, synthetic construct
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC